(19) United States
(12) Patent Application Publication
(10) Pub. No.: US 2012/0116271 A1
(43) Pub. Date: May 10, 2012

(54) CELLULITE TREATMENT

(75) Inventors: Joseph P. Caruso, Reading, MA (US); Andrei V. Belikov, St. Petersburg (RU); Gregory B. Altshuler, Lincoln, MA (US); James J. Childs, Bolton, MA (US)

(73) Assignee: PALOMAR MEDICAL TECHNOLOGIES, INC., Burlington, MA (US)

(21) Appl. No.: 13/325,028

(22) Filed: Dec. 13, 2011

Related U.S. Application Data
(63) Continuation-in-part of application No. 12/842,734, filed on Jul. 23, 2010.
(60) Provisional application No. 61/271,593, filed on Jul. 23, 2009.
(60) Provisional application No. 61/422,652, filed on Dec. 13, 2010.

(51) Int. Cl. A61H 7/00; A61B 18/18; A61B 18/04

(52) U.S. Cl. 601/6; 606/27; 606/32

(57) ABSTRACT

A method and apparatus are provided for treating connective tissue. The method and apparatus can be used for treating and/or reducing the appearance of cellulite.

The present application claims priority as a continuation-in-part to U.S. patent application Ser. No. 12/842,734, entitled “Method for Improvement of Cellulite Appearance” and filed Jul. 23, 2010, which claims priority to U.S. Patent Application Ser. No. 61/271,593, filed on Jul. 23, 2009 which are herein incorporated by reference in their entireties. This application also claims priority to U.S. Patent Application Ser. No. 61/422,652, filed on Dec. 13, 2010, which is herein incorporated by reference in its entirety.

### BACKGROUND OF THE INVENTION

The appearance of cellulite on a person's body can create a perception that the person is unfit and/or overweight. Individuals, generally women who have cellulite, often view it as unflattering and as a source of embarrassment. It is desirable to improve and/or eliminate the appearance of cellulite in one or more locations of a subject's body. It is most desirable to achieve a long term and/or durable improvement and/or to eliminate the appearance of cellulite in treated regions.

### SUMMARY OF THE INVENTION

In accordance with the methods and devices disclosed herein the invention generally relates to the treatment of connective tissue in a subject's body to improve the appearance of cellulite on a subject's body. In some embodiments, the methods and devices treat connective tissue with substantially lasting, durable and/or irreversible results. Long lasting, durable and/or irreversible treatment of connective tissue can improve the appearance of cellulite for a relatively long period of time and/or substantially permanently.

In one aspect, the invention relates to a method for improving the appearance of cellulite that comprises heating a portion of connective tissue to a temperature of at least about 50° C., and applying a tensile force to the heated connective tissue. In various aspects, the tensile force per unit area is greater than about 0.1 N/cm². In some aspects, the tensile force per unit area is greater than about 1 N/cm². In some aspects, the tensile force is sufficient to stretch the connective tissue. In some embodiments, the tensile force is sufficient to break the connective tissue. In various embodiments, the tensile force per unit area is insufficient to cause bruising of the skin.

Heating the connective tissue can be performed invasively or non-invasively in a variety of manners. For example, in various embodiments, the heating step can comprise applying energy to the portion of connective tissue through a skin surface. In a related aspect, the heating step can comprise applying at least one of optical energy, electrical energy, RF energy, and ultrasound energy to the connective tissue. In some embodiments, the heating step comprises applying optical energy having at least one wavelength in a range of about 600 nm to about 2700 nm to the connective tissue. For example, the optical energy can have at least one wavelength in a range of about 910 nm to about 930 nm (e.g., about 915 nm). In a related aspect, the optical energy can comprise a plurality of pulses, for example, pulses having a pulsewidth in a range of about 0.1 second to about 10 seconds. The optical energy can be produced by a variety of sources, for example, coherent sources such as a laser or laser diode or incoherent sources such as a lamp.

In one aspect, the heating step can comprise delivering a treatment tip through a skin surface to a location adjacent one or more septa. The treatment tip can be configured to deliver at least one of optical energy, electrical energy, RF energy, and ultrasound energy to the septa. By way of example, the heating step can comprise applying optical energy having at least one wavelength in a range of about 600 nm to about 2700 nm to the connective tissue, for example, at least one wavelength in a range of about 910 nm to about 930 nm (e.g., about 915 nm). In some aspects, the optical energy can comprise a plurality of pulses having, for example, a pulsewidth in a range of about 0.1 second to about 10 seconds.

In various embodiments, the connective tissue can comprise one or more septa. The tensile force can be sufficient to break at least a portion of said one or more septa in said heated connective tissue. In some embodiments, the connective tissue can comprise one or more septa comprising collagenous fibers and blood vessels associated therewith, wherein the tensile force is sufficient to break at least one or more collagenous fibers within the one or more septa.

In one aspect, the tensile force can be exerted on the connective tissue by applying suction to a skin surface. By way of example, applying suction to the skin surface can comprise disposing near the skin surface an element having a cavity formed therein for receiving a portion of skin tissue, said element having one or more passageways for applying an evacuative force to the cavity.

In various embodiments, the tensile force can be applied to the connective tissue at least one of during and after said heating step. In some embodiments, the target can comprise. In one aspect, the portion of connective tissue can be heated to a temperature in a range of about 50° C. to about 100° C. By way of example, the portion of connective tissue can be heated to a temperature in a range of about 50° C. to about 70° C.

In one aspect, the invention relates to a method for improving the appearance of cellulite. The method comprises positioning an element having a cavity formed therein adjacent skin tissue having a cellulite-mediated dimple, said element having one or more passageways for applying an evacuative force to the cavity. The method can also comprise activating a vacuum source so as to apply the evacuative force to draw a portion of the skin tissue into the cavity, the suction being effective to apply a tensile force to one or more septa within the skin. The method can also comprise heating a portion of the skin tissue to a temperature of at least about 50° C.

In various embodiments, the method can comprise inserting a treatment tip into the skin tissue and positioning the treatment tip adjacent the one or more septa. The method can also comprise delivering energy through the treatment tip to the one or more septa so as to heat said one or more septa. In some aspects, heating a portion of the skin tissue can comprise applying at least one of optical energy, electrical energy, RF energy, and ultrasound energy to the skin tissue.

In one aspect, the invention relates to a device for treating cellulite that comprises a vacuum source configured to generate a negative pressure. The device can also comprise a housing adapted to be placed in contact with a skin surface, the housing defining a cavity in fluid communication with the vacuum source through one or more passageways within the housing such that at least a portion of the skin tissue is drawn into the cavity when negative pressure generated by the source is applied to said cavity. The device also comprises an energy source configured to apply energy to said skin tissue disposed within the cavity so as to heat at least a portion of connective tissue to a temperature of at least about 50° C.

In various embodiments, the connective tissue comprises one or more septa, and the negative pressure can be configured to apply a tensile force greater than about 0.1 N/cm² to said one or more septa. For example, the tensile force per unit area can be greater that about 1 N/cm².

In various embodiments, the energy source can be configured to deliver at least one of optical energy, electrical energy, RF energy, and ultrasound energy. In one aspect, the energy source can be configured to deliver optical energy having at least one wavelength in a range of about 600 nm to about 2700 nm, for example, at least one wavelength in a range of about 910 nm to about 930 nm (e.g., 915 nm).

In one aspect, the device can also comprise a fluid flow pathway extending through the housing and in fluid communication with the vacuum source and the cavity. The fluid flow pathway can contain a liquid that is pumped by the vacuum source so as to generate the negative pressure in the cavity.

In one aspect, a device for treating cellulite is disclosed herein that includes a housing adapted to be placed in contact with a skin surface. The housing defines a cavity. A fluid flow pathway extends through the housing and is in fluid communication with the cavity and a source for generating a negative pressure on a liquid contained within the fluid flow pathway so as to draw at least a portion of the skin tissue into the cavity when negative pressure generated by the source is applied to said cavity.

In various embodiments, the fluid flow pathway can be in thermal contact with a cooling element. The cooling element can be configured to cool the liquid to a temperature in the range of about −5° C. to about 5° C., for example. In some embodiments, the fluid flow pathway can be in thermal contact with a heating element. The heat element can be configured to heat the liquid to a temperature in the range of about 35° C. to about 45° C., for example.

The negative pressure on the liquid can be at a variety of pressures. By way of example, the negative pressure on the liquid can comprise a pressure in the range of from about −0.1 bar to about −0.5 bar. For example, the negative pressure on the liquid can comprise a pressure in the range of from about −0.2 bar to about −0.3 bar.

In one aspect, the invention relates to a device for treating tissue that comprises an optical radiation source, and an optical fiber extending from a proximal end to a distal end and configured to emit from the distal end optical radiation generated by the radiation source. The device can also comprise a conductive heating element disposed at the distal end of the optical fiber, wherein the conductive heating element and the distal end of the optical fiber are disposed so as to define a cavity therebetween. The conductive heating element can be positioned relative to the fiber such that the conductive heating element is configured to receive optical radiation emitted from the distal end of the optical fiber so as to heat tissue in thermal contact with the conductive heating element.

In some aspects, the conductive heating element can comprise a rod extending along a length of the optical fiber. In a related aspect, the distal end of the rod can be disposed relative to the distal end of the optical fiber so as to define a concave cutting surface. In various embodiments, the conductive heating element can comprise a sleeve coupled to the optical fiber. The sleeve can comprise a plurality of protrusions extending distally beyond the distal end of the optical fiber. In some embodiments, the protrusions can be configured to engage tissue therebetween. In one aspect, the device can be configured to be inserted through the skin. The device can have, for example, a diameter at its distal end in a range of from about 1 mm to about 3 mm. In some aspects, the device can also comprise a vibration element configured to vibrate at least one of the distal end of the optical fiber and the heating element.

In one aspect, there is provided a device for improving the appearance of cellulite that comprises an optical radiation source; and an elongate probe extending from a proximal end to a distal end. The device also comprises an optical fiber coupled to the elongate probe and configured to emit from a distal end optical radiation generated by the radiation source. The distal end of the elongate probe can be configured to vibrate, for example, to ease the insertion of the probe through tissue. In some aspects, the distal end of the probe can be rounded. In various aspects, the distal end can vibrate in a range of from about 0.5 mm to about 2 mm at a frequency in a range from about 10 Hz to about 120 Hz.

### DESCRIPTION OF THE DRAWINGS

Further understanding of various aspects of the invention can be obtained by reference to the following description in conjunction with the associated drawings, which are described briefly below.

FIG. 1 is a schematic view of the inside of a subject's body in a region of cellulite; the schematic view depicts the subcutaneous tissue, which is located between the skin (e.g., the epidermis and dermis) and muscle and bone. The subcutaneous tissue includes a relatively thin layer (e.g., a single layer) of subcutaneous fat.

FIG. 2 is a schematic view of the inside of a patient's body in a region of cellulite; the schematic view depicting the subcutaneous tissue, which is located between the skin (e.g., the epidermis and dermis) and muscle and bone. The subcutaneous tissue includes a relatively thick layer (e.g., a multiple layers) of subcutaneous fat.

FIG. 3 shows a diagram of the generalized relationship of force applied to connective tissue on the x axis and the elongation of the connective tissue in response to the applied force on the y axis.

FIG. 4A depicts an experimental set-up for determining exemplary treatment parameters including the relationship between temperature of the tissue and the load applied.

FIG. 4B presents the results obtained by using the experimental set-up depicted in FIG. 4A.

FIG. 5 schematically depicts an exemplary embodiment of a device and method for treating and/or reducing the appearance of cellulite.

FIG. 6 presents the effect of the application of various wavelengths of optical radiation on the surface temperature of the skin.

FIG. 7 depicts another exemplary embodiment of a device and method for treating and/or reducing the appearance of cellulite.

FIG. 8A depicts another exemplary embodiment of a device and method for treating and/or reducing the appearance of cellulite.

FIG. 8B depicts another exemplary embodiment of a device and method for treating and/or reducing the appearance of cellulite.

FIG. 9 depicts another exemplary embodiment of a device and method for treating and/or reducing the appearance of cellulite.

FIG. 10 depicts another exemplary embodiment of a device and method for treating and/or reducing the appearance of cellulite.

FIG. 11A depicts an exemplary contour of the contact plate of FIG. 10.

FIG. 11B depicts another exemplary contour of the contact plate of FIG. 10.

FIG. 12 depicts an exemplary embodiment of a device and method for treating and/or reducing the appearance of cellulite.

FIG. 13 depicts an exemplary embodiment of a treatment probe that can be inserted into skin tissue for treating and/or reducing the appearance of cellulite.

FIG. 14 depicts another exemplary embodiment of a treatment probe that can be inserted into skin tissue for treating and/or reducing the appearance of cellulite.

FIG. 15 depicts another exemplary embodiment of a treatment probe that can be inserted into skin tissue for treating and/or reducing the appearance of cellulite.

### DETAILED DESCRIPTION

Anatomically, the cutaneous formation of cellulite is often due to fibrosis of the connective tissues present in the dermis and/or in the subcutaneous tissue. Connective tissue of the reticular dermis is connected to the deep fascia by fibrous septum from adipose or fat tissue. Subcutaneous fat lobules are separated from each other by fibrous septum (i.e., septa), which are generally relatively thin and usually rigid strands of connective tissue. The fibrous septa cross the fatty layer and connect the dermis to the underlying fascia tissue. The septa stabilize the subcutis and divide the fat tissue. Shortening of these septa due, for example, to fibrosis, causes retraction of the septa which in turn causes the depressions in the skin that are recognized as cellulite.

Thus, cellulite appears in the subcutaneous level of skin tissue where fat cells are arranged in chambers of fat tissue that are surrounded by bands of connective tissue called septae and/or fascia. Under certain conditions, for example, as water is retained, fat cells held within the perimeters of these fat tissue chambers expand and stretch the connective tissue. In some situations, the septa tissue is physiologically short and/or the septa tissue contracts and hardens holding the skin at a non-flexible length, while the surrounding tissue continues to expand with weight, or water gain, which results in areas of the skin being held down while other sections bulge outward, resulting in the lumpy, “cottage-cheese” appearance recognized as cellulite.

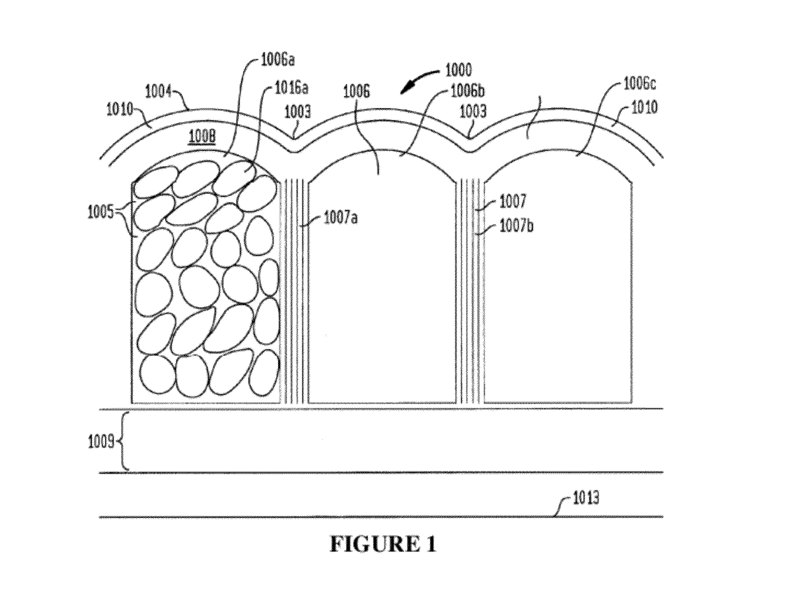

Referring now to FIG. 1, inside a subject's body 1000, between muscle 1009 and dermis 1008 is connective tissue called fiber stents or septa 1007. In some embodiments, bone 1013 is adjacent to muscle 1009. Fiber septa 1007 are bundles of connective tissue fibers that are held between the dermis 1008 and the muscle 1009. As discussed herein, fiber stents include soft tissue such as fibrous septa, which is composed of collagen fiber material similar to what is found in the dermis tissue, vascular tissue, and lymph tissue. Septa 1007 align and connect the muscle 1009 and the dermis 1008 to one another. The septa 1007 traverse through at least a portion of fat tissue 1006 inside the subject's body 1002. In some individuals, generally in females, when a volume of fat tissue 1006 between septa 1007 (e.g., between one septae 1007a and another septae 1007b) is over a threshold amount it creates an uneven, dimpled, and/or bumpy appearance on the external portion of the body 1004 and these dimples 1003 and/or bumps in the tissue are recognized as cellulite appearance. Cellulite appears due to the interaction of the existing fat 1006 with the septa 1007. A person with low fat could have cellulite because they have tight septa 1007. In some instances, cutting the septa 1007 in the region of the dimples 1003, e.g., in the areas between the bumps, with a knife to relieve the stress caused by the volume of fat tissue 1006 between septa 1007 (e.g., adjacent septa 1007a and 1007b) provides relief to the stress on the skin tissue that previously resulted in a dimpled and/or bumpy appearance. Cutting the septa 1007 can result in a flattening of the skin that was formerly bumpy in the region of the septa 1007. However, cutting the septa 1007 inside the skin is dangerous because it risks unintended consequences including nerve damage and muscle damage, for example.

Cellulite is generally a problem for females but is less common in males. In females the septa 1007 between the dermis 1008 and the muscle 1009 are substantially vertical relative to the plane of the dermis 1008 and/or the plane of the muscle 1009. Generally, the fibrous septa in women are orientated in a direction perpendicular to the cutaneous surface. In contrast, males have septa between the dermis and the muscle that are shifted to the side at an angle relative to the substantially vertical direction of the septa found in females. In males the septa have an angled or criss-cross pattern that does not feature the perpendicular direction relative to the cutaneous surface. Without being bound to a single theory, it is believed that the shifted angle of septa found in males provides a level of “give” such that changes in fat quantity inside a male's body do not result in the cellulite appearance. In addition, subcutaneous fat is divided into lobules and in women the fat lobules are relatively larger and more rectangular when compared with the fat lobules found in men. The substantially vertical septa 1007 found in females does not afford the “give” provided by the criss-cross pattern in males, further, the relatively larger size of fat lobules in women contribute to the cellulite appearance problem being more common for females than for males.

Thus, the substantially vertically oriented septa 1007 in females are primarily responsible for the typical orange peel/bumpy appearance that is recognized as cellulite. FIG. 1 depicts body areas having relatively thin subcutaneous fat (e.g., a single layer of fat tissue 1006) such as, for example, the under arms and the abdomen (i.e., the belly). The relative thickness or thinness of a body area will vary depending on individual anatomy.

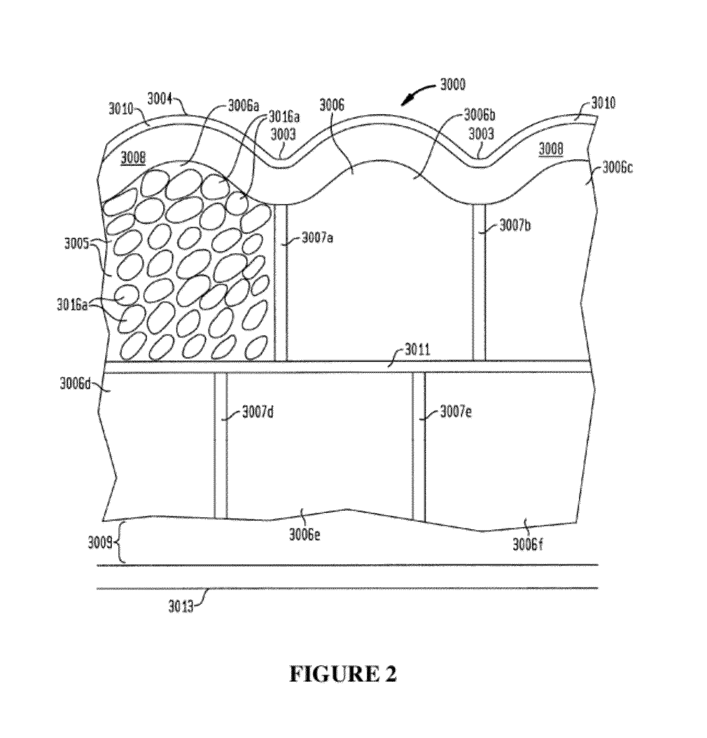

FIG. 2 shows a patient's body 3000, and more specifically, a body area having a relatively thick layer of subcutaneous fat made up of multiple chambers of fat tissue (e.g., 3006a, 3006b, 3006c, 3006d, 3006e, and 30060 some of which are stacked on one another (e.g., 3006b and 3006e). Relatively thick layers of subcutaneous fat that are made up of multiple chambers of fat tissue can include, for example, the buttocks and/or the thighs. The inside of a patient's body 3000 under the epidermis 3010, between muscle 3009 and dermis 3008 includes connective tissues including septa 3007 (also referred to as fiber stents) and fascia 3011. In some embodiments body areas that include cellulite have bone 3013 adjacent to muscle 3009.

Generally, a woman's anatomy features connective tissue including one or more vertical septa 3007 that are substantially vertical relative to at least one of the fascia 3011, the muscle 3009, and/or the skin (e.g., the epidermis 3010 and the dermis 3008). The septa 3007 traverse through at least a portion of fat tissue 3006 inside the subject's body 3002. Referring still to FIG. 2, in body areas having a relatively thick layer of subcutaneous fat, multiple layers of fat tissue 3006 are stacked between, above and below connective tissue. More specifically, inside the subject's body 3002 in the region of some body areas having a relatively thick region of subcutaneous fat, the fat tissue 3006 is stacked between substantially vertical septa 3007 and above and below substantially horizontal fascia 3011. In some embodiments, the fat tissue 3006 chambers (e.g., 3006a, 3006b, 3006c, 3006d, 3006e, and 30060 have an irregular pattern.

The connective tissue including the septa 3007 and the fascia 3011 align and connect the muscle 3009 and the dermis 3008 to one another. In some subjects, generally in females, when a volume of fat tissue 3006 between connective tissue 3007 (e.g., between one septa 3007b and another septa (e.g., 3007a and 3007d) and fascia 3011) is over a threshold amount it creates an uneven, dimpled, and/or bumpy appearance on the external portion of the body 3004 and these dimples 3003 and/or bumps in the tissue are recognized as cellulite appearance. Cellulite appears due to the interaction of the existing fat 3006 with the connective tissue (e.g., the septa 3007 and/or the fascia 3011). Without being bound to any single theory it is believed that in some embodiments, the fascia 3011 connects to the septa 3007 and acts as an anchor that holds the septa 3007 in a position that increases the pull of the septa 3007 against the dermis 3008 and/or the epidermis 3010 and this tension/pull contributes to the cellulite appearance provided by the dimples 3003.

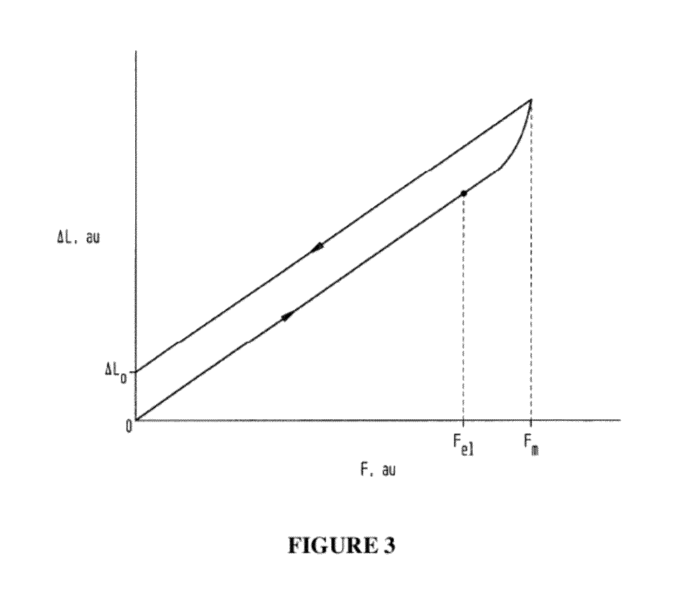

FIG. 3 is a diagram that shows the generalized relationship of force applied to connective tissue and the elongation of the connective tissue in response to the applied force. The force applied to connective tissue (e.g., septa and/or fascia) is shown on the x axis (force shown as F in arbitrary units (au)) and the y axis shows the elongation of the connective tissue (e.g., septa and/or fascia) as ΔL (in arbitrary units). The x axis also shows Fₘ which is the elasticity limit of the connective tissue being treated. The elasticity limit is the maximum force which provides a change in length ΔL of the connective tissue that is directly proportional to the applied force F. The x axis also shows Fₘ, which is the maximum force applied during a given elongation treatment. The y axis shows ΔLo, which is the lasting elongation after releasing the force F applied to the connective tissue. Lasting elongation includes elongation that lasts for several hours after treatment, e.g., two or more hours after treatment, and can include elongation that is substantially irreversible (i.e., elongation that is maintained and is substantially permanent) after treatment.

As seen in FIG. 3, when the maximum force Fₘ is higher than the elasticity limit Fₑ₁ then elongation of the connective tissue becomes non-linear such that it responds to an applied force that is greater than Fₑ₁ in a non-linear manner. After releasing the applied force F the length of the connective tissue demonstrates hysteresis behavior (as described in greater detail in reference to FIG. 3A of U.S. Ser. No. 12/842,734, which is incorporated by reference herein), which results in the lasting elongation having the quantity depicted as ΔLo. The Fₑ₁ can be a function of the tissue temperature and the time of application of the temperature to tissue. By elevating tissue temperature, the Fₑ₁ may be lowered and the lasting elongation ΔLo can be achieved with a relatively lower Force than is required in the absence of an elevated temperature. Thus, by increasing the temperature of the connective tissue to be treated with a force F, the amount of force required to improve the length of (e.g., elongate) the connective tissue is reduced. In this way, negative side effects to the body area being treated including tearing, bruising and pain can be reduced and/or avoided.

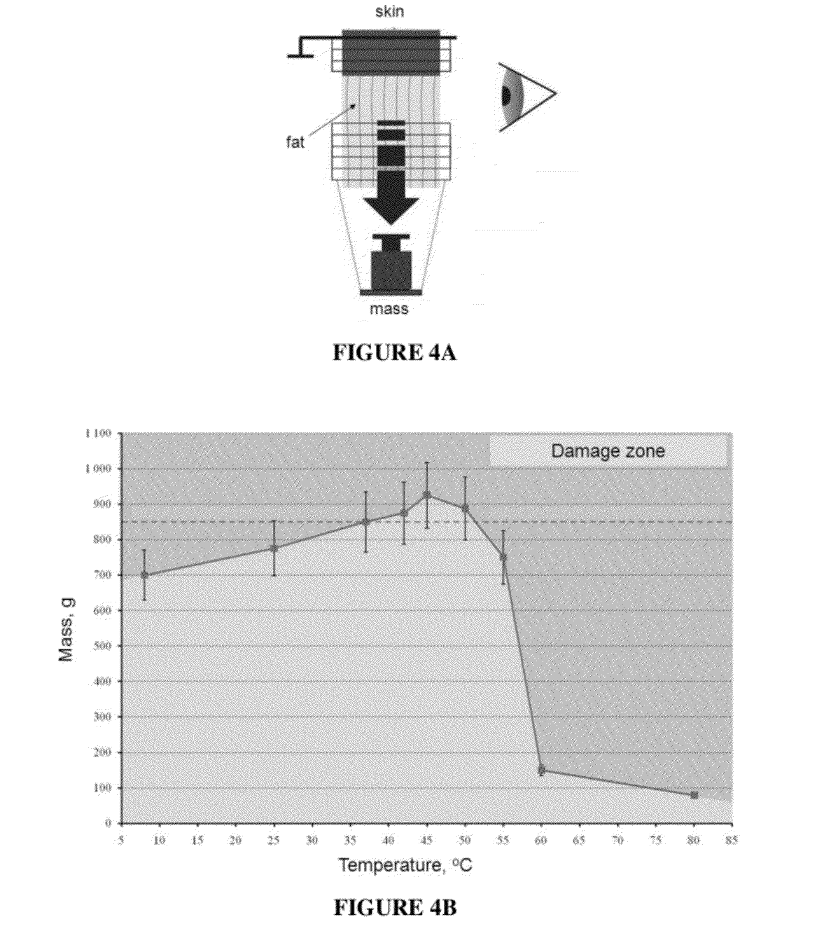

FIG. 4A depicts an experimental set-up for determining exemplary treatment parameters. As shown in FIG. 4A, a sample of porcine skin (e.g., the dermis and subcutaneous fat) can be used to determine treatment parameters. The fat of the tissue sample can be coupled to a mass to test the relationship between the tensile load applied to the sample and the temperature of the sample.

FIG. 4B presents the results obtained for porcine skin by using the experimental set-up of FIG. 4A. The plot of FIG. 4B shows that the “tensile strength” of the samples (the mass in grams that the sample can withstand) substantially and drastically decreases at temperatures above about 50° C. The data presented herein shows that the amount of force needed to break the connective tissue can be substantially reduced when the skin tissue is at temperatures of at least about 50° C., or from about 50° C. to about 100° C., or from about 50° C. to about 70° C.

The treatment parameters (e.g., the energy and tensile load applied to the tissue) are preferably selected to minimize, and preferably eliminate, undesired damage to the tissue, for example, bruising of the patient. Accordingly, methods and devices disclosed herein can be configured to improve the appearance of cellulite (e.g., by breaking fibrous septa), while preventing excessive or undesired tissue damage and/or bruising.

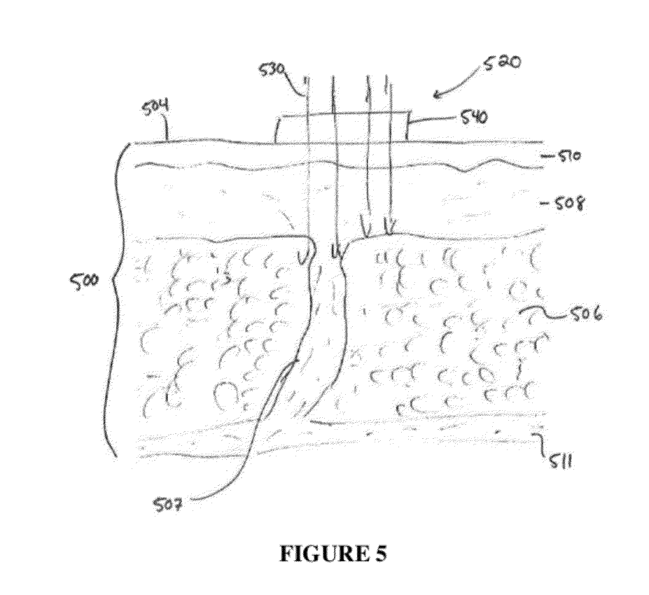

The temperature of the skin tissue can be elevated in order to reduce the force necessary to break connective tissue and/or remodel the skin tissue using a variety of devices and methods in accord with the teachings herein. By way of example, energy can be delivered to the skin tissue invasively, for example via a probe inserted through an incision, or non-invasively, for example through the external application of energy. With reference now to FIG. 5, an exemplary embodiment of a device 520 for non-invasively treating and/or improving the appearance of cellulite is shown. Though the device 520 is depicted as delivering optical energy 530 to heat at least a portion of the skin tissue 500 through the skin surface 504, it will be appreciated by a person skilled in the art that the device 520 can instead or additionally be configured to deliver one or more of radiofrequency (RF) energy, ultrasonic energy, microwave energy, or thermal energy (e.g., via thermal conduction) through the skin surface 504 in order to heat the subcutaneous tissue to temperatures at which the force of breaking the connective tissue is reduced. As shown in FIG. 5, the device 500 can deliver optical energy 530 to the subcutaneous fat 506, for example, through the skin surface 504 to heat the septa 507 attached to the lower portion of the dermis 508. An optical window 540, which can be made of a material (e.g., sapphire) having high thermal conductivity and a refractive index to aid in coupling the optical energy into the skin tissue 500, can be placed in contact with the skin surface 504. The optical energy 530 applied through the optical window 540 can heat an entire region of skin tissue 500 in which the target tissue is located and/or preferentially heat a target tissue at depth. By way of example, optical energy 530 that is selectively absorbed by the skin tissue 500 below the level of the dermis 510 (e.g., subcutaneous fat) can be applied to the skin surface 504. In use, as the septa is heated by the optical energy to temperatures in a range from about 40° C. to about 65° C., the tension on the septa 507, which causes the dimple/cellulite appearance, can be sufficient to break the septa 507. As will be discussed in detail below, in various embodiments, additional tension can be applied to the septa 507 concurrent with or subsequent to heating to break the septa 507, for example, through the application of a vacuum.

The optical energy 530 can be generated by a variety of sources. For example, any of coherent, incoherent, continuous, and/or pulsed sources of optical energy can be used with the device 520. In various embodiments, diode or solid state lasers and filtered arc lamps can be used to generate the optical energy. The optical sources can be contained within the device 520, for example, or can be operatively coupled thereto. In some embodiments, optical radiation in a wavelength range of from about 0.8 microns to about 1.6 microns, preferably from about 910 to about 930 nm and/or from about 1200 nm to about 1220 nm, and in a power density range of from about 20 to about 7000 W/cm² can be generated by a source and pass through the optical window 540. In various embodiments, pulses of the optical energy can be applied to the skin tissue 500 for time periods ranging from about 1 second to about 20 seconds. Optical radiation can be delivered in one beam or in multiple separated micro-beams (e.g., fractional micro-beams).

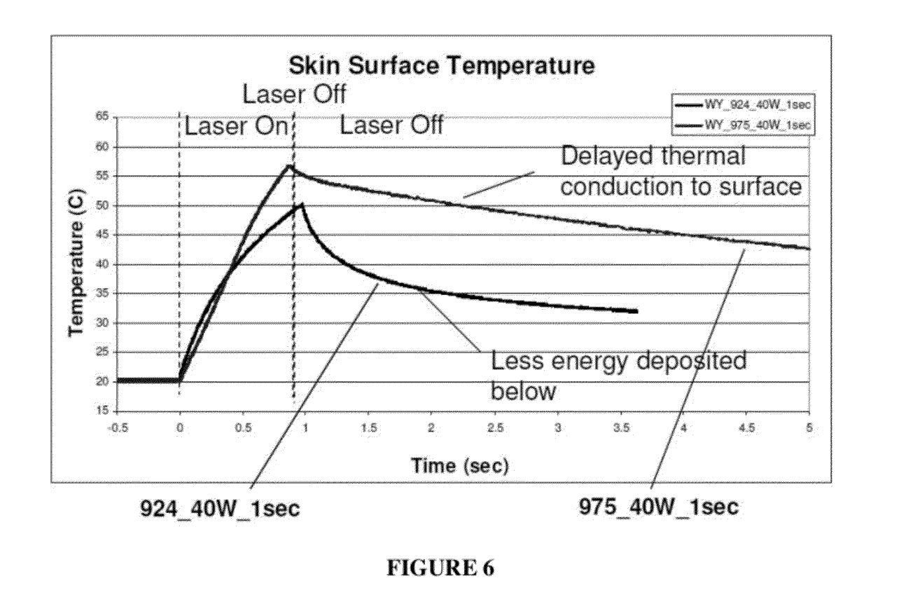

Referring now to FIG. 6, experimental data resulting from the application of various wavelengths of optical radiation to skin tissue is shown. As shown in FIG. 6, the delivery of optical radiation to the skin tissue 500 can raise the temperature of the skin tissue during and subsequent to irradiation by various light sources. By way of example, FIG. 6 demonstrates that the delivery of optical energy having a wavelength of 924 nm and at a power of 40 W can be effective to raise the temperature of the skin surface to about 50° C. within about one second. Likewise, the delivery of optical energy having a wavelength of 975 nm and at 40 W can be effective to raise the temperature of the skin surface to about 55° C. within about one second. After terminating the application of the radiation, the skin surface temperature can decrease at a rate depending on the rate of thermal conduction from tissue at depth. By way of example, the rapid cooling of the skin surface following the application of optical energy having a wavelength of 924 nm relative to that of skin surface following the application of optical energy having a wavelength of 975 nm indicates that the 924 nm optical energy provides deeper penetration into the skin tissue. The data also suggest that less energy is deposited immediately below the skin surface by optical radiation having a wavelength of 924 nm relative to that of optical energy having a wavelength of 975 nm.

Though the wavelength of the optical radiation can be selected so as to target a tissue at depth (e.g., subcutaneous fat), FIG. 6 indicates that the temperature of the skin surface can be raised through thermal conduction from the targeted tissue. To reduce skin surface heating, which can reduce pain experienced by a patient undergoing treatment, contact cooling of the skin surface can be provided. With reference again to FIG. 5, the device 520 can be configured to cool the surface of the skin before, during, or after the delivery of optical energy thereto. By way of example, the optical window 540 can be configured to remove heat from the surface of the skin. By way of example, the optical window 540 can be in thermal contact with a cooling element coupled to the device 520. By way of non-limiting example, a thermoelectric Peltier cooler can be used to cool the optical window 540. Alternatively, the optical window 540 can include channels containing coolant. In various embodiments, the channels containing the coolant can thermally contact the edge of the optical window 540 so as not to obstruct viewing and/or delivery of optical energy 530 therethrough. The optical window 540 can be maintained at various temperatures to provide sufficient contact cooling of the skin surface. By way of example, the optical window 540 can be maintained at a temperature in a range of from about −5° C. to ambient temperature, preferably from about 0° C. to about 18° C., to maintain the temperature of the entire dermis and epidermis of the skin at temperatures between about 0° C. and 42° C. In various embodiments, optical energy 530 can be delivered to the skin tissue 500 prior to contact cooling, concurrent with contact cooling, and/or subsequent to contact cooling.

In one aspect, methods for the noninvasive treatment of the appearance of cellulite can also include cyclically heating and cooling the skin tissue, or alternatively, simply cooling the skin tissue to remodel the skin tissue 500 in accord with the teachings herein. By way of example, the optical window 540 can be operated as a cooling plate that can cool the skin tissue to a depth, and through which optical energy can be applied intermittently as discussed in U.S. Pat. No. 7,276,058, which is herein incorporated by reference in its entirety, and modified in accord with the teachings herein.

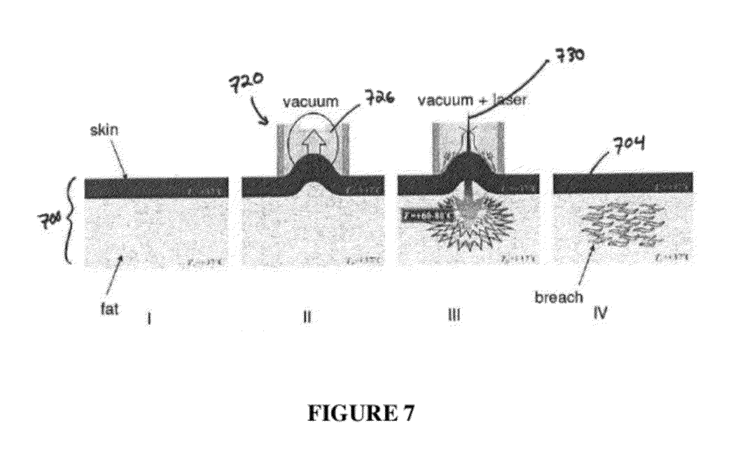

Reference now is made to FIG. 7, which depicts an exemplary method and device for remodelling the skin. As shown in FIG. 7, a device 720 can be located adjacent the skin and a vacuum can be applied to a cavity 726 of the device 720 when the device 720 is placed in contact with the skin surface 704. The vacuum can be effective to draw the skin tissue 700 into the cavity 726 and apply a tensile load on the skin tissue 700. For example, the suction can be effective to provide a tensile load per unit area less than about 10 N/cm². In one aspect, the suction can provide a tensile force per unit area of between about 0.1 N/cm² to about 10 N/cm², and more preferably in a range of about 0.1 N/cm² to about 1 N/cm². In another aspect, the suction can provide a tensile force per unit area greater than about 0.1 N/cm². By way of example, the tensile force can be greater than about 1 N/cm², greater than about 2 N/cm², greater than about 5 N/cm², greater than about 5 N/cm², or greater than about 10 N/cm². In various embodiments, the tensile force can sufficient to stretch or break the connective tissue.

After engaging the skin tissue 700 within the cavity 726, energy (e.g., optical energy 730) can be applied to the skin tissue contained therein to heat the skin tissue 700, and preferably, the subcutaneous skin tissue. By raising the temperature to a range of about 50° C. to about 100° C. (e.g., in a range of about 60° C. to about 80° C.), while applying the suction to the skin tissue 700, subcutaneous connective tissue can be altered as otherwise discussed herein. By way of example, septa present in the subcutaneous tissue can be stretched and/or broken. Additionally or in the alternative, the application of energy to the skin tissue 700 can be effective to remodel the structure of the skin, which can lead to thickening of the dermal layer, for example. In such a manner, the device 720 can be effective to treat and/or reduce the appearance of cellulite using a non-invasive means. Though the method described above demonstrates the application of optical energy 730, it should be appreciated that other forms of energy such as electrical energy, radiofrequency (RF) energy, and ultrasound energy can also be applied to the skin tissue in accord with the teachings herein.

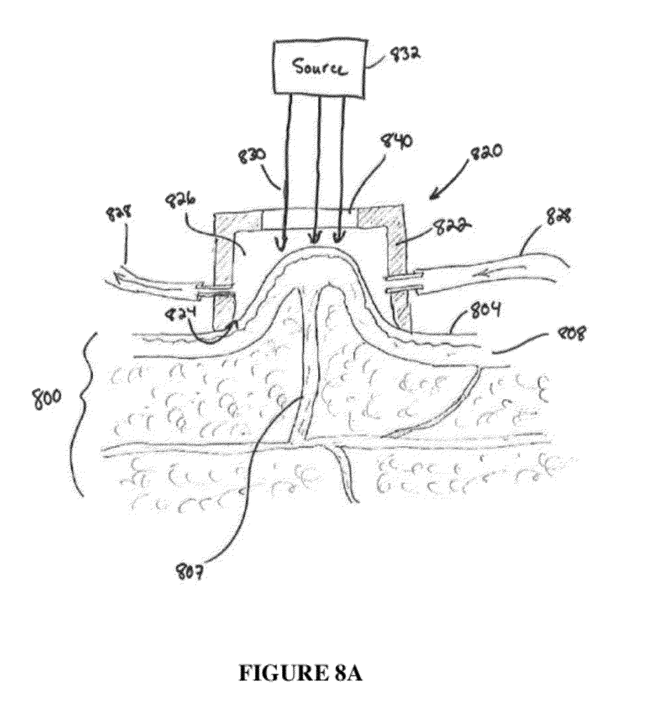

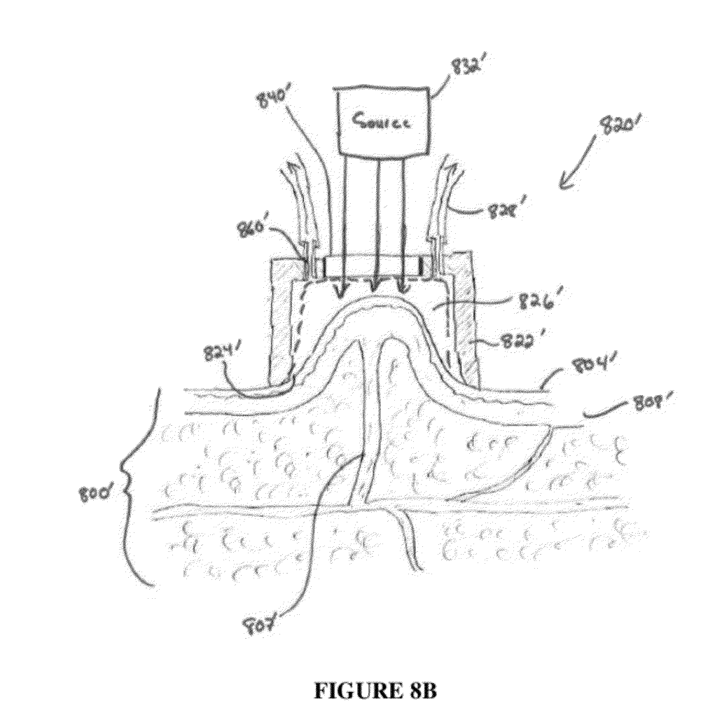

With reference now to FIG. 8A, another embodiment of a method and device for non-invasively treating and/or improving the appearance of cellulite is shown. As otherwise discussed herein, the device 820 can be configured to provide a stretching force to the skin tissue 800 while applying energy (e.g., optical energy 830) thereto. As shown in FIG. 8, the device 820 can include a suction cup 822 having an open end 824 that can be applied to the skin surface 804 such that a portion of the skin tissue 800 can be positioned within cavity 826 when a negative pressure is applied thereto. At least a portion of the suction cup 822 can be optically transparent, for example optical window 840, such that optical energy can be applied to the skin tissue 800 contained within the cavity 826. The suction cup 822 can be coupled to a vacuum pump (not shown) that can be operated to draw air out of the cavity 826 through conduits 828. By way of example, the vacuum pump can reduce the pressure in the cavity 826 to a pressure in the range of from about 100 to about 500 Torr, preferably from about 200 to about 380 Torr when the open end 824 of the suction cup 822 is placed in contact with the skin surface 804. This sub-atmospheric pressure can draw the skin tissue 800 into the cavity 826, thereby stretching the dermis 808 and septa 807 that is attached thereto. As discussed elsewhere herein, by placing the device 820 over a cellulite dimple and applying a negative pressure thereto before, during, and/or after delivery of energy to the skin tissue 800, the septa 807 responsible for the cellulite dimple can be stretched and/or broken to treat and/or improve the appearance of cellulite. By way of example, one or more pulses of optical energy 830 can be delivered to the skin tissue 800 disposed within the cavity 826 that can be sufficient to heat the septa 807 causing it to break. The optical energy 830 can be generated by a variety of sources, as discussed otherwise herein. In various embodiments, a source 832 (e.g., a diode or solid state laser, filtered arc lamp) can be used to generate the optical energy. The source 832 can be contained within the device 820, for example, or can be operatively coupled thereto (e.g., from a base unit).

With reference now to FIG. 8B, another exemplary embodiment of a method and device for non-invasively treating and/or improving the appearance of cellulite is shown. The device 820′ is substantially similar to that described above in reference to FIG. 8A. For example, the device 820′ can include a suction cup 822′ having an open end 824′ that can be applied to the skin surface 804′ such that a portion of the skin tissue 800 can be positioned within cavity 826′ when a negative pressure is applied thereto. Conduits 828′, however, can be fluidly coupled to the cavity 826′ through passageways 828′ that extend through the suction cup 822′ around the perimeter of the optical window 840′. By positioning the passageways 860′ adjacent or in proximity to the optical window 840′ (e.g., in an annular ring around the circumference of the window), application of a negative pressure to the cavity 826′ can be effective to draw the skin tissue 800′ into the cavity such that the skin surface can be in contact with the optical window 840′, for example, as shown by the dashed line. As such, optical radiation generated by the source and directed through the optical window 840′ can be optically coupled directly into the skin tissue 800′, rather than being transmitted through the cavity 826′. As will be appreciated by a person skilled in the art, the optical window can comprise a material with a similar refractive index to that of skin to further aid in optically coupling the radiation into the skin.

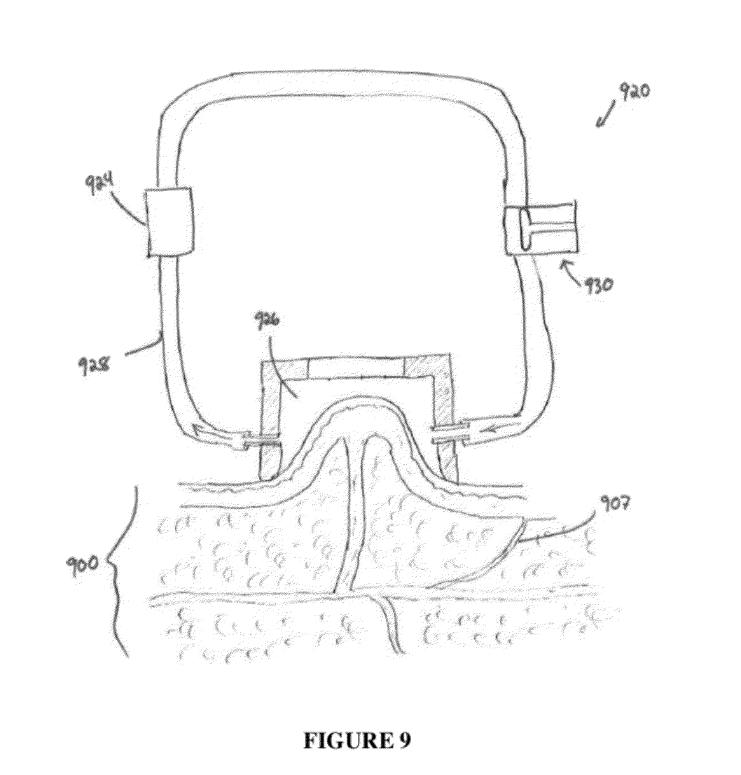

With reference now to FIG. 9, another exemplary embodiment of a method and device for non-invasively treating and/or improving the appearance of cellulite is shown. The device 920 is similar to that of FIG. 8A. However, whereas the conduits 828 can be fluidly coupled to a vacuum source operable to evacuate gas from within the cavity 826 to draw the skin tissue 800 therein, a liquid can be pumped through the fluid flow pathway 928 to apply a negative pressure to the cavity 926 to draw the skin tissue 900 within the cavity. As shown in FIG. 9, the fluid flow pathway 928 can be associated, for example, with any of a pump 930, a cooling element 932, and/or a heating element (not shown). By way of example, the device for applying negative pressure to the liquid, such as a pump 930 (e.g., piston), can be configured to apply a sufficient negative pressure to the liquid within the fluid within cavity 926 to draw the skin tissue 900 into the cavity 926. For example, actuation of the pump 930 can be effective to apply a negative pressure to the liquid in the cavity 926, e.g., to apply a pressure in the range from about −0.1 bar to about −0.5 bar, thereby drawing the tissue 900 into the cavity 926. In some aspects, actuation of the pump 930 can be effective to apply a pressure in the range from about −0.2 bar to about −0.3 bar. As will be appreciated by a person skilled in the art, one or more valves can be provided to control the flow of fluid through the fluid flow pathway and/or into and out of the cavity 926. It was unexpectedly discovered that, in accord with various aspects of the methods and systems disclosed herein, sufficient suction could be generated by applying a negative pressure to a liquid contained within the cavity 926 to draw the skin tissue 900 into the cavity 926. Further, as will be discussed in detail below, the use of a cooling liquid in the flowing fluid pathway was found to be efficient in regulating the temperature (e.g. cooling) of the tissue. Without being bound by any particular theory, it is believed that the application of suction to the tissue within the cavity can promote increased blood flow to the skin, which is cooled by the liquid in the cavity. As the cooled blood flows to deeper tissue, it can facilitate cooling of that deeper tissue. Hence, the combination of suction and cooling of the skin can advantageously provide efficient cooling of deep tissue. Such cooling can in some embodiments reduce, or eliminate, the sensation of pain, e.g., as energy, such as optical energy, is applied to the connective tissue.

With continued reference to FIG. 9, in one aspect, the liquid supplied by the fluid flow pathway 928 can be effective to cool or heat the skin tissue 900. By way of example, a cooling or heating element 924 (e.g., a heat exchanger, thermoelectric element such a Peltier cell, etc.) can be provided to cool and/or heat the fluid flowing through the fluid flow pathway 928. In some aspects, the cooling or heating liquid can be pumped through the fluid flow pathway into and out of the cavity 926 at temperatures in the range of from about −5° C. to about 5° C. or from about 35° C. to about 45° C., respectively, As will be appreciated by a person skilled in the art, one or more auxiliary pumps can also be associated with the fluid flow pathway 928 to circulate the fluid contained therein, even under the increased pressure provided by the pump 930. In some embodiments, the heating and cooling fluid can be applied in a cyclical fashion so as to cyclically heat and cool the skin tissue 900 in the area of the dimple.

In various embodiments, after a period of cooling and/or heating, for example in the range of from about 10 minutes to about 45 minutes, one or more pulses of optical radiation can be delivered to the skin tissue 900 to further heat the septa 907, thereby causing them to stretch and/or break. As described above, the optical energy can have a wavelength in a range of from about 0.8 microns to about 1.6 microns, preferably from about 910 to about 930 nm and/or from about 1200 to about 1220 nm, and a power density in a range of from about 20 to about 7000 W/cm². In various embodiments, pulse(s) of the optical energy 930 can be applied to the skin tissue 900 for a time duration ranging from about 1 second to about 20 seconds.

In some embodiments, cooling and/or heating fluid can be applied in a cyclical fashion so as to cyclically heat and cool the skin tissue 900 (e.g., fat cells) in the area of the dimple. Additionally, application of a cooling fluid can be alternated with heating of the skin tissue 900 through the delivery of optical energy 930. While heating or cooling alone can be useful for many treatments, heating and cooling applied intermittently to the skin surface (e.g., contrast therapy) can provide beneficial effects in reducing subcutaneous fat deposits and/or treating or improving the appearance of cellulite, as generally described in detail in U.S. Pat. No. 7,276,058, which is herein incorporated by reference in its entirety, and modified in accord with the teachings herein.

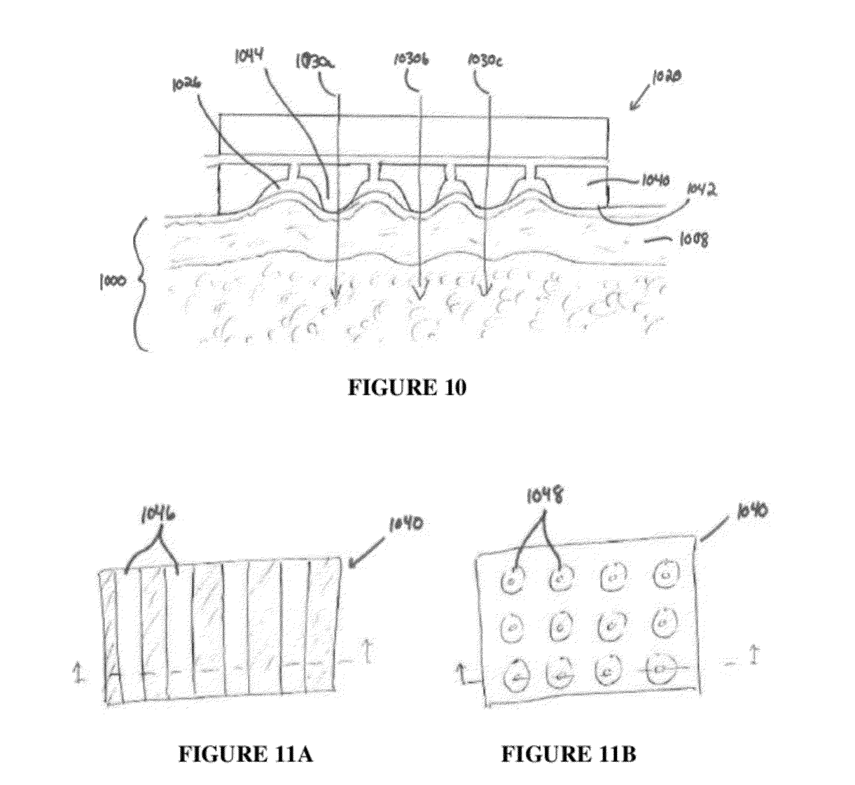

Referring now to FIG. 10, an embodiment of a device and method for the noninvasive treatment of the appearance of cellulite is shown. As otherwise discussed herein, the device 1020 can provide stretching of the skin tissue 1000 (and its underlying connective tissue including the dermis 1008 and septa 1007). By way of example, a contact plate 1040 having a contoured skin-contacting surface 1042 can be placed in contact with the skin surface 1004. The contact plate 1040 can have a variety of configurations to provide a contoured skin-contacting surface 1042. By way of example, the protuberances 1044 of the contact plate 1040 can provide compression and stretching of the skin 1000. With reference now to FIGS. 11A and 11B, which depict exemplary embodiments of a contact plate having the cross-section depicted in FIG. 10 (along the dotted lines of FIGS. 11A and 11B), the skin-contacting surface 1042 of the contact plate 1040 can include multiple grooves 1046 (e.g., a sinusoidal groove pattern as shown in FIG. 11A) or a plurality of separated dimples 1048 (e.g., an array of dimples as shown in FIG. 11B).

As discussed otherwise herein, sub-atmospheric pressure can be applied through ports 1048 in the contact plate 1040 to draw the skin into the contact plate's recesses 1026 disposed between the protuberances 1044. By way of example, a vacuum supply (not shown) can be operatively coupled to the ports 1028 to reduce the pressure in the recesses 1026 to a pressure in the range of from about 100 to about 500 Torr, preferably from about 200 to about 380 Torr. Likewise, the contact plate 1040 can be configured to provide contact cooling and/or heating of the skin tissue 1000 as discussed above. For example, contact plate 1040 can be cooled by inter-laced cooling lines or thermo-electric elements.

In addition, optical radiation can be applied to the skin tissue 1000 through the contact plate 1040. As shown in FIG. 10, for example, the optical energy can be delivered as discrete, spatially separated beams (e.g., micro-beams 1030a-c). By way of example, each of the micro-beams 1030a-c can be delivered through the contact plate 1004 to the skin tissue 1000 through a protuberance 1044 to heat the dermis and/or subcutaneous fat beneath the protuberance 1044 by photothermolysis. By virtue of the multiple micro-beams 1030a-c, in some embodiments, the dermis 1008, for example, can coagulate only the position which receives the micro-bean 1030a-c, thereby creating a fractional pattern of coagulation. Accordingly, in some embodiments, the device 1020 can provide fractional stretching, cooling, and irradiation of the skin tissue 1000. As a result of the fractional coagulation of the skin tissue, the healing process of the fractionally-treated skin tissue, as discussed generally in U.S. Pat. No. 6,997,923, can be effective to thicken the dermis, thereby improving the appearance of cellulite.

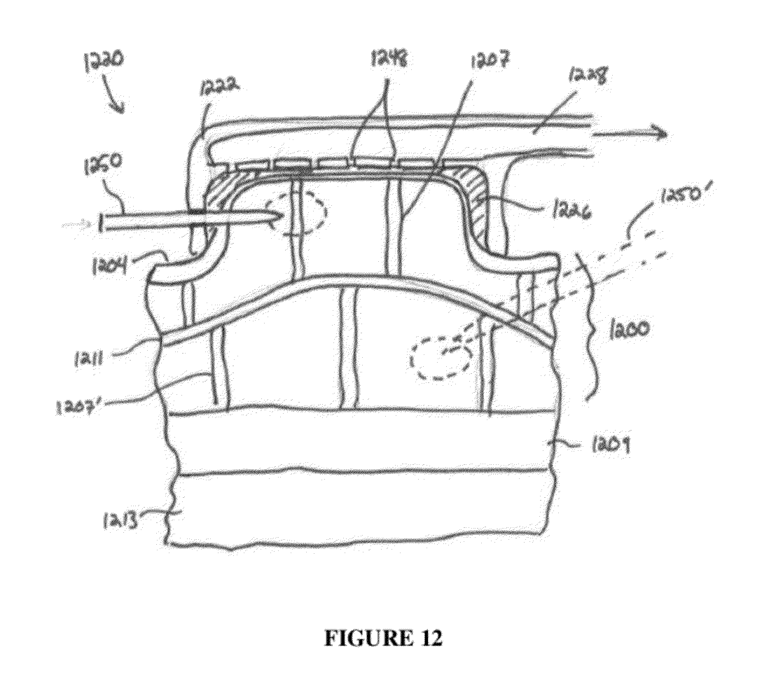

With reference now to FIG. 12, an exemplary embodiment of a device for treating and/or reducing the appearance of cellulite is shown. The device 1220 can include a housing 1222 (e.g., a handpiece) for contacting the skin surface 1204. The housing 1222 can define a cavity 1226 therein that is configured to receive and/or engage at least a portion of skin tissue 1200 including the tissue that underlies the skin surface 1204. As depicted, the skin surface 1204 overlays a subcutaneous fat layer having a substantially vertical septa 1207 therethrough, a layer of fascia 1211, another layer of subcutaneous fat having substantially vertical septa 1207′ disposed therethrough, a muscular layer 1209, and bone 1213.

The housing 1222 can additionally include a passageway 1228 that can connect the cavity 1226 to a vacuum pump (not shown), such as an aspirator vacuum pump. One or more holes 1248 can provide fluid communication between the passageway 1228 and the cavity 1226 such that activation of the vacuum pump can be effective to apply suction to at least a portion of a skin surface 1204 and underlying tissue to draw the tissue into the cavity 1226. The suction of the skin tissue 1200 can be effective to apply a tensile load on the skin tissue 1100 and the associated septa 1207 and/or 1207′. In one aspect, the suction can be effective to provide a tensile load per unit area less than about 10 N/cm². In one aspect, the suction can provide a tensile force per unit area of between about 0.1 N/cm² to about 10 N/cm², and more preferably in a range of about 0.1 N/cm² to about 1 N/cm². In another aspect, the suction can provide a tensile force per unit area greater than about 0.1 N/cm². By way of example, the tensile force can be greater than about 1 N/cm², greater than about 2 N/cm², greater than about 5 N/cm², greater than about 5 N/cm², or greater than about 10 N/cm². In various embodiments, the tensile force can sufficient to stretch or break the connective tissue.

As shown in FIG. 12, the device 1220 can also include a treatment tip v50 that can be configured to heat a portion of the skin tissue 1200 disposed within the cavity 1226. By way of example, a sidewall of the cavity 1226 can include an opening 1227 that allows the treatment tip 1250 to be inserted into the tissue (e.g., via access provided by an incision) disposed within the cavity 1226. Alternatively, as shown in phantom by the treatment probe 1250′, a sidewall of the cavity need not include an opening. Rather, the treatment probe 1250 can be inserted directly into the skin and can be positioned adjacent, for example, a target tissue under tensile force caused by the application of a vacuum, as discussed otherwise herein. At least a portion of the treatment tip 1250 can be positioned adjacent a septa 1207 and energy can be applied to the tissue to cause localized heating thereof. As will be appreciated by a person skilled in the art, any mechanism for heating the tissue can be effective to heat at least a portion of the skin. By way of non-limiting example, the treatment tip 1250 (e.g., an end of the tip 1250) can be configured to apply optical energy (e.g., laser or other light emission), electrical energy (ohmic resistance), RF energy, microwave energy or ultrasound energy. By way of example, these energy sources can have a power level from about 1 watt to about 100 watts, or from about 10 watts to about 60 watts.

In one embodiment, the treatment tip 1250 can be configured to heat a portion of the tissue to at least 50° C. For example, the tissue (e.g., septa 1207 and surrounding tissue as indicated by the dashed line) can be heated to a temperature in a range of about 50° C. to about 100° C. (e.g., in a range of about 50° C. to about 70° C.). The treatment tip 1250 can be used, for example, to apply one or more pulses of optical energy to the tissue. The one or more pulses can have at least one wavelength in a range of between about 800 nm to about 11 microns. For example, the optical energy can have at least one wavelength in a range of 800 nm to about 3 microns, in a range of about 910 nm to about 930 nm, or about 915 nm. In some embodiments, optical energy can have at least one wavelength in the range of from about 0.8 microns to about 1.6 microns, preferably from about 910 to about 930 nm or from about 1200 to about 1220 nm. One or more of the pulses can also have a pulsewidth in a range of about 0.1 second to about 10 seconds. In some aspects, the treatment tip 1250 can provide a conduit for passage of an optical fiber so that the tip of the fiber can be positioned in proximity to the connective tissue under treatment for application of radiation thereto.

In some aspects, at least a portion of the element 1222 can be transparent, for example, to allow a user to position the device over a desired area of the skin to be treated (e.g. a cellulite dimple). Thus, a user could mark a cellulite-mediated dimple, for example, and align the element 1222 over the mark on the skin surface (e.g., the cellulite dimple). Additionally, in one aspect, energy can be applied directly through a transparent portion of the element 1222. In one embodiment the treatment tip 1250 is a laser that includes an aiming beam. Because in this illustrative embodiment, at least a portion of the element 1222 is transparent, the user can visualize the location of the treatment tip 1250 by its aiming beam and its position relative to the marked cellulite-mediated dimple. In this way the user can ensure that the region of the tissue beneath the surface of the dimple (e.g., at least a portion of substantially vertical septa 1207, 1207′) is heated in the presence of vacuum.

Referring still to FIG. 12 in a non-invasive embodiment, instead of using the invasive treatment tip 1250, an energy source (not shown) that is external to the skin may be employed to heat a portion of the tissue, as discussed above with reference to FIGS. 5, 7, and 8A. For example, the tissue (e.g., septa 1207 and surrounding tissue as indicated by the dashed line) can be heated to a temperature in a range of about 50° C. to about 100° C. (e.g., in a range of about 50° C. to about 70° C.) using a non-invasive means. Suitable energy sources employed during the heating step can include, for example, focused ultrasound. In one embodiment, the heating step includes applying energy to the portion of skin tissue through a surface of the skin using at least one of optical energy, electrical energy, radiofrequency (RF) energy, and ultrasound energy to the skin tissue.

In an embodiment where at least a portion of the element 1222 is transparent the element can be made from, for example, a transparent resin. The element can be reusable, disposable (e.g., designed for a one-time use) or substantially long lasting. In one embodiment, the cavity 1226 has a diameter measuring from about 0.5 inches to about 10 inches and has a depth of from about 0.5 inches to about 5 inches.

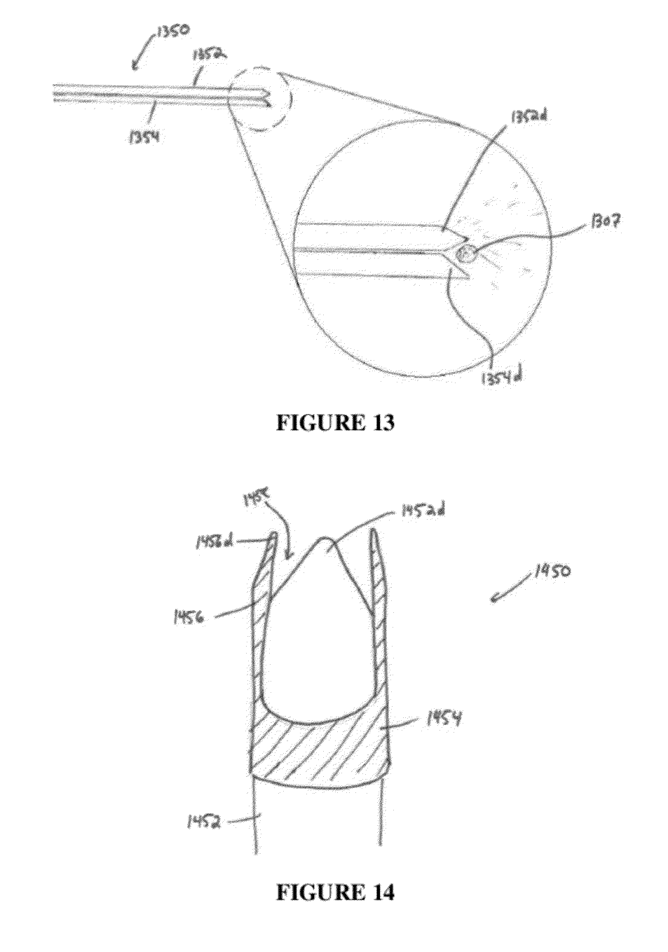

Referring now to FIG. 13, an exemplary embodiment of a treatment probe 1350 for treating and/or improving the appearance of cellulite is depicted. While the treatment probe 1350 can be used in conjunction with the device 1220 as discussed above with reference to the treatment tip 1250 depicted in FIG. 12, the treatment probe 1350 can also be inserted directly into tissue to cut, for example, a septa connecting the dermis with underlying fascia. As shown in FIG. 13, the treatment probe 1350 can include a light-delivery fiber 1352 that is configured to deliver optical energy from its distal tip 1352d. The fiber 1352 can be optically coupled to an optical energy source (not shown), for example, a diode laser or solid-state laser. In one aspect, the source can generate optical energy having at least one wavelength in the range of from about 900 to about 1300 nm, preferably from about 910 to about 930 nm, and can have a power from about 20 W to about 70 W. In some aspects, pulses can range from about 1 to about 3 seconds to deliver from about 20 to about 210 Joules of optical energy.

The treatment probe 1350 also includes a rod 1354 that extends at least partially along a length of the fiber 1352. The rod 1354 can be positioned relative to the distal tip 1352d of the fiber 1352 such that it can receive at least a portion of the optical energy emitted by the fiber 1352. The rod 1354 is generally configured to be heated upon irradiation by the fiber 1352 and can be formed from a variety of materials and can be rigid, semi-rigid, or flexible. By way of example, the rod 1354 can comprise metal. Though the rod 1354 is shown having a similar diameter to that of the fiber and extending along the entire length of the fiber 1352, a person of skill in the art will appreciate that the rod 1354 can have various configurations that enable its use in a treatment probe 1350 as discussed herein. By way of example, rather than extending along the entire length of the fiber 1352, the rod 1354 may extend only along the distal end of the fiber 1352.

The distal ends of the fiber 1352 and rod 1354 can be disposed relative to one another so as to define a substantially concave cutting surface 1356 between the distal ends. In one aspect, optical energy (e.g., generated by a laser) that is emitted from the fiber tip 1352d can heat the distal end 1354d of the rod 1354 to an elevated temperature, e.g., a temperature sufficient to cut and/or sever connective tissue. Additionally, optical energy emitted by fiber 1352 can be effective to heat the septa 1307 such that the force necessary to cut, sever, or tear the septa 1307 is decreased relative to that required under normal physiologic temperatures.

In use, the treatment probe 1350 can be inserted through a small incision in the skin and positioned at a target region (e.g. septa) located beneath the skin surface 1304. By way of example, the treatment probe 1350 can be disposed beneath the dermis-hypodermis junction to engage a septa 1307 extending between the fascia and the dermis. In one aspect, the treatment probe 1350 can be advanced so as to dispose the substantially concave cutting surface 1356 adjacent a target tissue (e.g., septa 1307). One or more pulses of optical energy generated by a source can be delivered through the fiber 1352 and emitted at its distal tip 1352d. The optical energy can be sufficient to heat the distal tip 1354d of the rod 1354 as well as the septa 1307 that is positioned in thermal contact therewith. For example, the optical energy and/or the heated distal end 1354d of the rod 1354 can be effective to heat the septa 1307 at or near the temperature of coagulation. Concurrent with or subsequent to heating, a force can be applied to break the septa. By way of example, the treatment probe 1350 can be advance towards the septa 1307.

With reference now to FIG. 14, another exemplary embodiment of a treatment probe in accord with various aspects of applicants' teachings is depicted. As shown in FIG. 14, the treatment end (e.g., the distal end) of the treatment probe 1450 can include a light-delivery fiber 1452 that can be optically coupled to an optical energy source and can be configured to deliver optical energy from its distal tip 1452d. The distal tip 1452d can have a variety of configurations and can comprise a variety of materials through which the optical energy can be emitted. By way of non-limiting example, the distal tip can comprise sapphire or quartz. Though the distal tip 1452d is depicted with a tapered configuration, it will be appreciated that the tip can have a variety of shapes, for example, flat, recessed, etc.

The treatment probe 1450 also includes a sleeve 1454 that removably of fixedly coupled to the distal end of the fiber 1452. By way of example, the sleeve 1454 can circumferentially surround the distal end of the fiber 1453. It should be appreciated that the sleeve 1454 can extend proximally along the fiber 1452 for various lengths, for example, the entire length of the fiber 1452 to a position outside the body when the treatment probe 1450 is disposed therein. As shown in FIG. 14, the sleeve 1454 can include one or more protrusions 1456 that extend distally from the sleeve 1454. The protrusions 1454 can extend at least partially around the distal-most end 1452d of the fiber 1452 and can have various lengths. By way of example, the distal-most ends of the protrusions 1354 can be substantially level with the distal-most end 1452d of the fiber 1452. Alternatively, the distal-most ends 1456d can extend beyond the distal-most end of the fiber 1452. In various aspects, the sleeve 1454 an/or protrusions 1456 can be positioned relative to the distal tip 1452d of the fiber 1452 such that it can receive at least a portion of the optical energy emitted by the fiber 1452 and can be heated upon irradiation by the fiber 1452. The sleeve 1454 can be formed from a variety of materials and can be rigid, semi-rigid, or flexible. By way of example, the rod 1454 can comprise a metal such as stainless steel. Additionally, the protrusions 1456 can be disposed relative to one another and the fiber so as to define a cavity 1457 for receiving a target tissue (e.g., septa). In one aspect, optical energy (e.g., generated by a laser) that is emitted from the fiber tip 1452d can heat the protrusions 1456 to an elevated temperature, e.g., a temperature sufficient to cut and/or sever connective tissue. Additionally, optical energy emitted by fiber 1452 can be effective to heat the target tissue within the cavity 1457 such that the force necessary to cut, sever, or tear the tissue is decreased relative to that required under normal physiologic temperatures. In various aspects, the probes described herein (e.g., probe 1450) can have a diameter at their distal end in a range from about 1 mm to about 3 mm.

In use, the treatment probe 1450 can be operated in a similar matter as discussed above with reference to the treatment probe 1350 depicted in FIG. 13. For example, the treatment probe 1450 can be inserted through a small incision in the skin and positioned at a target region (e.g. septa) located beneath the skin surface. By way of example, the treatment probe 1450 can be disposed beneath the dermis-hypodermis junction and can be advanced so as to dispose a target tissue (e.g., septa) between the protrusions 1456 extending distally from the sleeve 1454. One or more pulses of optical energy generated by a source can be delivered through the fiber 1452 and emitted at its distal tip 1452d. The optical energy can be sufficient to heat the sleeve 1454 and/or its protrusions 1456 as well as the septa, for example, that is positioned in thermal contact therewith. The optical energy and/or the sleeve 1454 and/or the protrusions 1456 can be effective to heat the septa at or near the temperature of coagulation. Concurrent with or subsequent to heating, a force can be applied to break the septa. By way of example, the treatment probe 1450 can be advance towards the septa.

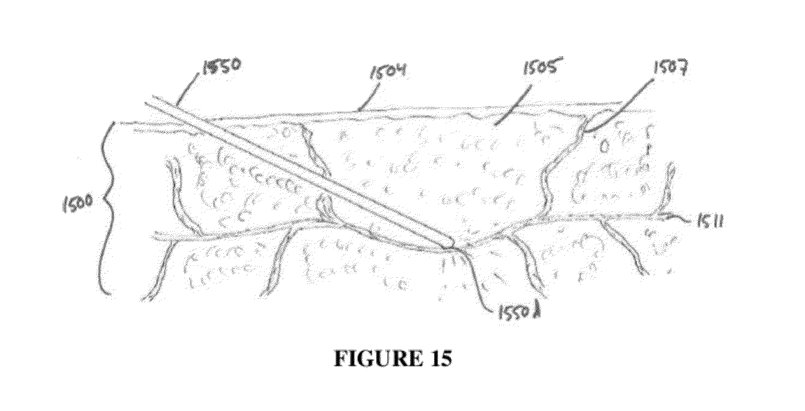

With reference now to FIG. 15, a treatment probe 1550 for treating and/or improving the appearance of cellulite through the targeted heating of the fascia 1511 is depicted. The treatment probe 1550 is configured to be inserted through the skin surface and can be advanced such that the distal tip 1550d is disposed below the dermis-hypodermis junction. The probe 1550 itself or a light-fiber coupled to or extending through the treatment probe 1550 can be configured to deliver optical energy from its distal tip 1550d directly to the underlying superficial or deep fascia (such as Camper's fascia or Scarpa's fascia). The distal tip 1550d can have a variety of configurations to ease its movement through tissue. For example, the tip 1550d can be tapered so as to reduce frictional force. In one aspect, the distal-most end of the treatment probe 1550 can be rounded to prevent accidental damage. Additionally or in the alternative, in some embodiments, the distal tip 1550d can be configured to vibrate to reduce frictional forces experienced by the tip 1550d and to ease motion through subcutaneous tissue such as fat 1505 and septa 1507. A rounded distal tip 1550d and vibration of the distal end of the treatment probe can reduce the risk of perforating the fascia 1511 such that the tip can “ride” on the fascia 1511 without penetrating therethrough.

By way of example, the probe 1550 can be optically coupled to a source (not shown) such as a diode laser or solid-state laser that is configured to generate optical energy. In one aspect, the source can generate optical energy that can be applied to the target tissue (e.g., fascia 1511) having at least one wavelength in the range of from about 900 to about 1300 nm, preferably from about 910 to about 975 nm, and can have a power from about 20 W to about 70 W. The source can be operated in continuous mode or in pulsed mode. In one aspect, the pulses can have a pulse width from about 0.1 to about 2 seconds at repetition rates from about 0.5 Hz to about 5 Hz.

In use, as shown in FIG. 15, the treatment probe 1550 can be inserted through an incision in the tissue and can be guided through the subcutaneous spaces to the target fascia 1511. Once the distal tip 1550d contacts the target fascia 1511, for example, the source can be activated such that optical energy coupled into the probe 1450 can be emitted from the distal tip 1550d. The tip 1550d can be moved during laser emission to heat the target fascia to stimulate contraction and new collagen growth, as otherwise discussed herein. In one aspect, if the user encounters resistance, for example, vibration can be activated to ease the motion of the distal tip 1550d through the tissue. In one aspect, the amplitudes of vibration of the distal tip 1550d can range from about 0.5 to about 2 mm at frequencies from about 10 to about 120 Hz.

Though the treatment probe 1550 of FIG. 15 is depicted as being inserted directly through the skin, one of skill in the art will appreciate that the treatment probe can also be inserted into the skin through a device configured to apply vacuum to the skin, as discussed above with reference to FIG. 12.

Though the devices discussed above are primarily described in their use for heating fascia, septa, or other subcutaneous tissue, it should be appreciated that these devices and methods can be applied to various portions of the skin for the treatment or improvement in the appearance of the skin. By way of example, a relatively thin dermal layer can also cause the appearance of cellulite. The devices and techniques described above can be modified to treat the appearance of cellulite through the thickening of the dermis, for example. As such, the application of energy to the dermal layer using the methods and devices described herein can be effective to stimulate new collagen growth and a thickening of the dermis.

What is claimed is:

1. A method for improving the appearance of cellulite, comprising:
heating a portion of connective tissue to a temperature of at least about 50° C.,
applying a tensile force to the heated connective tissue.

2. The method of claim 1, wherein said tensile force per unit area is greater than about 0.1 N/cm².

3. The method of claim 1, wherein said tensile force per unit area is greater than about 1 N/cm².

4. The method of claim 1, wherein the tensile force is sufficient to stretch the connective tissue.

5. The method of claim 1, wherein the tensile force is sufficient to break the connective tissue.

6. The method of claim 1, wherein said heating step comprises applying energy to the portion of connective tissue through a skin surface.

7. The method of claim 6, wherein said heating step comprises applying at least one of optical energy, electrical energy, RF energy, and ultrasound energy to the connective tissue.

8. The method of claim 7, wherein said heating step comprises applying optical energy having at least one wavelength in a range of about 600 nm to about 2700 nm to the connective tissue.

9. The method of claim 8, wherein said optical energy has at least one wavelength in a range of about 910 nm to about 930 nm.

10. The method of claim 8, wherein said optical energy has a wavelength of about 915 nm.

11. The method of claim 8, wherein said optical energy comprises a plurality of pulses having a pulsewidth in a range of about 0.1 second to about 10 seconds.

12. The method of claim 8, wherein said optical energy is produced by a laser.

13. The method of claim 1, wherein said heating step comprises delivering a treatment tip through a skin surface to a location adjacent one or more septa, wherein said treatment tip is configured to deliver at least one of optical energy, electrical energy, RF energy, and ultrasound energy to the septa.

14. The method of claim 13, wherein said heating step comprises applying optical energy having at least one wavelength in a range of about 600 nm to about 2700 nm to the connective tissue.

15. The method of claim 14, wherein said optical energy has at least one wavelength in a range of about 910 nm to about 930 nm.

16. The method of claim 14, wherein said optical energy comprises a plurality of pulses having a pulsewidth in a range of about 0.1 second to about 10 seconds.

17. The method of claim 1, wherein said connective tissue comprises one or more septa and wherein said tensile is sufficient to break at least a portion of said one or more septa in said heated connective tissue.

18. The method of claim 1, wherein said connective tissue comprises one or more septa comprising collagenous fibers and blood vessels associated therewith, wherein said tensile force is sufficient to break at least one or more collagenous fibers within the one or more septa.

19. The method of claim 1, wherein said tensile force is exerted on the connective tissue by applying suction to a skin surface.

20. The method of claim 19, wherein applying suction to the skin surface comprises disposing near the skin surface an element having a cavity formed therein for receiving a portion of skin tissue, said element having one or more passageways for applying an evacuative force to the cavity.

21. The method of claim 1, wherein said tensile force is applied to the connective tissue at least one of during and after said heating step.

22. The method of claim 1, wherein the portion of connective tissue is heated to a temperature in a range of about 50° C. to about 100° C.

23. The method of claim 1, wherein the portion of connective tissue is heated to a temperature in a range of about 50° C. to about 70° C.

24. A method for improving the appearance of cellulite, comprising:
positioning an element having a cavity formed therein adjacent skin tissue having a cellulite-mediated dimple, said element having one or more passageways for applying an evacuative force to the cavity,
activating a vacuum source so as to apply the evacuative force to draw a portion of the skin tissue into the cavity, the suction being effective to apply a tensile force to one or more septa within the skin, and
heating a portion of the skin tissue to a temperature of at least about 50° C.

25. The method of claim 24, further comprising:
inserting a treatment tip into the skin tissue,
positioning the treatment tip adjacent said one or more septa, and
delivering energy through the treatment tip to said one or more septa so as to heat said one or more septa.

26. The method of claim 24, wherein heating a portion of the skin tissue comprises applying at least one of optical energy, electrical energy, RF energy, and ultrasound energy to the skin tissue.

27. A device for treating cellulite, comprising:
a vacuum source configured to generate a negative pressure;
a housing adapted to be placed in contact with a skin surface, the housing defining a cavity in fluid communication with the vacuum source through one or more passageways within the housing such that at least a portion of the skin tissue is drawn into the cavity when negative pressure generated by the source is applied to said cavity; and
an energy source configured to apply energy to said skin tissue disposed within the cavity so as to heat at least a portion of connective tissue to a temperature of at least about 50° C.

28. The device of claim 27, wherein the connective tissue comprises one or more septa, and wherein the negative pressure is configured to apply a tensile force greater than about 0.1 N/cm² to said one or more septa.

29. The device of claim 27, wherein the energy source is configured to deliver at least one of optical energy, electrical energy, RF energy, and ultrasound energy.

30. The device of claim 27, wherein the energy source is configured to deliver optical energy having at least one wavelength in a range of about 600 nm to about 2700 nm.

31. The device of claim 30, wherein the optical energy has at least one wavelength in a range of about 910 nm to about 930 nm.

32. The device of claim 27, further comprising a fluid flow pathway extending through the housing and in fluid communication with the vacuum source and the cavity, the fluid flow pathway containing a liquid that is pumped by the vacuum source so as to generate said negative pressure in the cavity.

33. A device for treating cellulite, comprising:
a housing adapted to be placed in contact with a skin surface, the housing defining a cavity;
a fluid flow pathway extending through the housing and in fluid communication with the cavity and a source for generating a negative pressure on a liquid contained within said fluid flow pathway so as to draw at least a portion of the skin tissue into the cavity when negative pressure generated by the source is applied to said cavity.

34. The device of claim 33, wherein the fluid flow pathway is in thermal contact with a cooling element.

35. The device of claim 34, wherein the cooling element is configured to cool the liquid to a temperature in the range of about −5° C. to about 5° C.

36. The device of claim 33, wherein the fluid flow pathway is in thermal contact with a heating element.

37. The device of claim 36, wherein the heating element is configured to heat the liquid to a temperature in the range of about 35° C. to about 45° C.

38. The device of claim 33, wherein the negative pressure on the liquid comprises a pressure in the range of from about −0.1 bar to about −0.5 bar.

39. The device of claim 38, wherein the negative pressure on the liquid comprises a pressure in the range of from about −0.2 bar to about −0.3 bar.

40. A device for treating cellulite, comprising:
an optical radiation source;
an optical fiber extending from a proximal end to a distal end and configured to emit from the distal end optical radiation generated by the radiation source at the proximal end; and
a conductive heating element disposed at the distal end of the optical fiber, wherein the conductive heating element and the distal end of the optical fiber are disposed so as to define a cavity therebetween,
wherein the conductive heating element is positioned relative to the fiber such that the heating element is configured to receive optical radiation emitted from the distal end of the optical fiber so as to increase heat tissue in thermal contact with the conductive heating element.

41. The device of claim 40, wherein the conductive heating element comprises a rod extending along a length of the optical fiber,
a distal end of the rod being disposed relative to the distal end of the optical fiber so as to define a concave cutting surface.

42. The device of claim 40, wherein the conductive heating element comprises a sleeve coupled to the optical fiber.

43. The device of claim 42, wherein the sleeve comprises a plurality of protrusions extending distally beyond the distal end of the optical fiber,
the protrusions being configured to engage tissue therebetween.

44. A device for improving the appearance of cellulite, comprising:
an optical radiation source;
an elongate probe extending from a proximal end to a distal end; and
an optical fiber coupled to the elongate probe and configured to emit from a distal end optical radiation generated by the radiation source at the proximal end; and
wherein the distal end of the elongate probe is configured to vibrate.
